(19) United States
(12) Patent Application Publication
(10) Pub. No.: US 2022/0251019 A1
(43) Pub. Date: Aug. 11, 2022

(54) METHOD FOR PRODUCING GLYCEROL MONO(METH)ACRYLATE

(71) Applicant: EVONIK OPERATIONS GMBH, Essen (DE)

(72) Inventors: Sebastian BESTGEN, Echborn (DE); Maik CASPARI, Alsbach-Haehnlein (DE); Thorben SCHÜTZ, Alsbach-Hähnlein (DE); Tim BLEITH, Mainz (DE)

(73) Assignee: EVONIK OPERATIONS GMBH, Essen (DE)

(21) Appl. No.: 17/620,710

(22) PCT Filed: Jun. 18, 2020
(86) PCT No.: PCT/EP2020/066865

§ 371 Date: Dec. 19, 2021

(30) Foreign Application Priority Data
Jun. 21, 2019 (EP) 19181573.7

(51) Int. Cl. C07C 67/297; C07C 69/54

(52) U.S. Cl. CPC C07C 67/297; C07C 69/54

(57) ABSTRACT

The invention relates to a method for producing glycerol mono(meth)acrylate from 2,2-dimethyl-1,3-dioxolan-4-ylmethyl (meth)acrylate by acid-catalysed reaction with methanol.

### FIELD OF THE INVENTION

The invention relates to an anhydrous method for producing glycerol mono(meth)acrylate by reacting 2,2-dimethyl-1,3-dioxolan-4-ylmethyl (meth)acrylate with methanol.

### PRIOR ART

Glycerol mono(meth)acrylate can be used for various industrial applications, for example as a monomer for producing polymers for coating purposes, varnishes, paints, adhesives or contact lenses, as well as in the field of oil-based lubricants and slip additives.

For many applications it is critical that glycerol mono(meth)acrylate is in the form of high-purity monomer since even trace impurities can firstly result in negative effects during storage of the monomer and these secondly also impair the quality of the polymer to be produced.

Particularly undesirable impurities in monomers are those which may act as crosslinkers in a polymerization reaction since the presence of crosslinkers during a polymerization reaction impairs the formation of linear polymers. Even traces of acids considerably reduce the stability of the monomer.

Glycerol mono(meth)acrylate can, in principle, be obtained by various routes.

Firstly, glycerol can be esterified directly with acetylating reagents (e.g. methacrylic acid, methacrylic anhydride, methacryloyl halides). For example, WO 2018/031373 describes the preparation of glycerol monomethacrylate (GMMA) from glycerol and an excess of methacrylic acid in the presence of amberlyst. However, in this case, low selectivity is problematic.

More selective with respect to monofunctionalization is the mostly acid-catalysed epoxide and acetonide cleavage of glycidyl methacrylate or 2,2-dimethyl-1,3-dioxolan-4-ylmethyl methacrylate (IPGMA) in aqueous medium to give GMMA, wherein the cleavage of the harmful to health glycidyl methacrylate leads to the formation of undesired by-products (J. Appl. Polym. Sci., 135, 46579) or requires a laborious column chromatographic purification (Macromolecules 2018, 51, 18, 7396-7406).

The preparation of GMMA by hydrolysis of IPGMA is described, for example, in GB 852 384, wherein the hydrolysis is carried out in the presence of dilute aqueous solutions of strong mineral acids.

In US 2010/249352, the acetonide is cleaved in aqueous medium by addition of an acid, and is then neutralized with a weak base. The water is removed in a laborious process of multiple distillation on a thin-film evaporator.

The preparation of GMMA from IPGMA is also described in DE102016122755. The preparation is also carried out here in aqueous medium in the presence of amberlyst and with simultaneous feed of an O₂/N₂ gas stream.

GMMA can also be obtained via lipase-catalysed transacylation (J. Mol. Cat. B: Enzymatic 2010, 62, 80-89), which requires utilization of enzymes and long reaction times (120 h) and has low conversions.

The methods described above have the addition of water in common, which is very difficult to remove from the remaining reaction mixture/product. The consequence is a high residual water content in the product, which has a negative effect on the further use of glycerol mono(meth)methacrylate or can even completely prevent it. In addition, polymerization occurs during the dewatering phase owing to the prolonged thermal stress.

Particularly for the application of glycerol mono(meth)acrylate in the field of oil-based lubricants and slip additives, water can also be included among the particularly undesirable impurities. In the field of lubricants and hydraulic oils—in addition to contamination with solid particles—contamination with water or moisture can be the cause of component failure.

Water may mix with oil in different ways. At low concentrations, it can be dissolved in oil in the liquid phase and thus may not be visually noticeable (solubility in the range of several hundred ppm, material-dependent and age-dependent): It takes the form of a one-phase system. On further increasing the water content, the solubility limit is exceeded and (microscopically) small droplets are formed in the oil, which is known as an emulsion. The water content has in this case exceeded the saturation point, which results in a distinct and undesirable cloudiness of the oil. On further increasing the water content, this eventually results in the formation of two phases, namely a low-oil water phase and a low-water oil phase.

In the sector of lubricants and hydraulic oils, particularly free water and emulsified water have an adverse effect on the material characteristics, for example on the compressibility. Even low levels of impurities in the range of 1% can result in drastic shortening of the service life of the oil. In moving bearings, the formation of a stable oil film is prevented by water contamination. Furthermore, under high temperature and pressure load, there can be spontaneous evaporation of the water (and thus oil), which results in erosive wear. Oil contamination by water further causes foam formation, oil hydrolysis, metal embrittlement, rust and corrosion.

For the use of glycerol mono(meth)acrylate in oil-based systems, the aim is therefore the lowest possible water content in glycerol mono(meth)acrylate, which cannot be achieved for this hydrophilic monomer by means of current synthetic methods.

The object of the invention, therefore, was to provide an improved method for producing glycerol mono(meth)acrylate, by means of which the problems described above with respect to selectivity and by-product formation can be overcome, in which no toxic starting materials have to be used and in which no laborious dewatering step is required. Ideally, the glycerol mono(meth)acrylate product also should not comprise any traces of acid.

By means of this method, a crosslinker-free and the purest possible glycerol mono(meth)acrylate should be obtained without the use of mutagenic substances.

Moreover, the method should be achievable on an industrial scale.

### SUMMARY OF THE INVENTION

The object is achieved in that glycerol mono(meth)acrylate is produced by a silicate catalysed cleavage of 2,2-dimethyl-1,3-dioxolan-4-ylmethyl (meth)acrylate with methanol.

Accordingly, the method relates to a method for producing glycerol mono(meth)acrylate, particularly glycerol monomethacrylate (GMMA), characterized in that 2,2-dimethyl-1,3-dioxolan-4-ylmethyl (meth)acrylate, especially 2,2-dimethyl-1,3-dioxolan-4-ylmethyl methacrylate (IPGMA), is reacted with methanol in the presence of an acidic silicate catalyst.

In this manner, anhydrous glycerol mono(meth)acrylate is obtained without using glycidyl methacrylate that is harmful to health. By avoiding an initial water input to the reaction mixture, a time- and energy-intensive removal of water from the end product is avoided. Also, a prolonged dewatering phase and thus accompanying polymerization of the monomer is prevented.

### DETAILED DESCRIPTION OF THE INVENTION

The invention relates to both glycerol monomethacrylate (GMMA) and glycerol monoacrylate (GMA). Accordingly, the term glycerol mono(meth)acrylate, as used in the context of this invention, includes both GMMA and GMA.

The ratio by weight of 2,2-dimethyl-1,3-dioxolan-4-ylmethyl (meth)acrylate to methanol in the method according to the invention should be in the range from 1:1 to 1:20, preferably from 1:1 to 1:15 and particularly preferably in the range from 1:1 to 1:4.

For the reaction, both technical-grade methanol and high purity methanol with a purity of 99.9% can be used.

Particularly suitable silicate catalysts include clay minerals such as montmorillonite, kaolinite, hectorite, halloysite or mixtures thereof, for example bentonite. Particular preference is given to using acidic sheet silicates and aluminium silicates, such as Tonsil® 312 FF or montmorillonite K10, K30 etc. The catalysts used are particularly particles with the greatest possible specific surface area, particularly with specific surface areas greater than 50 m²/g, preferably with a specific surface area greater than 220 m²/g, and particularly preferably with a specific surface area greater than 320 m²/g.

Compared to homogeneous-catalysed acetonide cleavage of 2,2-dimethyl-1,3-dioxolan-4-ylmethyl (meth)acrylate with p-TsOH (para-toluenesulfonic acid) or the heterogeneous-catalysed acetonide cleavage of 2,2-dimethyl-1,3-dioxolan-4-ylmethyl (meth)acrylate with acidic ion exchange resins (Amberlyst®), an exceptionally low-crosslinker glycerol mono(meth)acrylate product is obtained particularly when using silicate-based catalysts: contamination with glycerol di(meth)acrylate is less than 1%. The product is also virtually free of free glycerol (<1%), which is also found in greater amounts in conventional synthetic routes.

Preferably, the reaction is performed in anhydrous manner and the product is anhydrous glycerol mono(meth)acrylate.

The acidic silicate catalyst is used in an amount of 0.5% by weight to 20% by weight, preferably in an amount of 1% by weight to 15% by weight, and especially in an amount of approximately 10% by weight, based on the reaction batch.

Pre-treatment of the catalyst is generally not required.

For stabilizing the starting material and/or product, stabilizers/polymerization inhibitors may be used.

Preferred polymerization inhibitors that can be used include, inter alia, octadecyl-3-(3,5-di-tert-butyl-4-hydroxyphenyl) propionate, phenothiazine, hydroquinone, hydroquinone monomethyl ether, 4-hydroxy-2,2,6,6-tetramethylpiperidinooxyl (TEMPOL), 2,4-dimethyl-6-tert-butylphenol, 2,6-di-tert-butylphenol, 2,6-di-tert-butyl-4-methylphenol, para-substituted phenylenediamines such as for example N,N′-diphenyl-p-phenylenediamine, 1,4-benzoquinone, 2,6-di-tert-butyl-alpha-(dimethylamino)-p-cresol and 2,5-di-tert-butylhydroquinone. These compounds can be used individually or in the form of mixtures and are generally commercially available. The mode of action of the stabilizers is usually that they act as free-radical scavengers for the free radicals that occur in the polymerization. Further details can be found in the relevant technical literature, particularly Römpp-Lexikon Chemie; publisher: J. Falbe, M. Regitz; Stuttgart, New York; 10th edition (1996); keyword “Antioxidantien” and the literature references cited therein.

The total amount of stabilizers used is between 0.0001% by weight and 0.5% by weight, preferably in the weight range between 0.001% by weight and 0.05% by weight.

Preferably, the stabilizers hydroquinone monomethyl ether and hydroxy-2,2,6,6-tetramethylpiperidinooxyl (TEMPOL) are used in combination. The ratio of hydroquinone monomethyl ether to TEMPOL is ideally in the range between 15:1 and 1:1, preferably in the range between 10:1 and 4:1.

In addition, gaseous oxygen can be used for stabilization. This can, for example, be in the form of air, in which the amounts introduced should be adjusted such that the content in the gas phase above the reaction mixture remains below the explosion limit.

The reaction times depend, inter alia, on the selected parameters such as pressure and temperature. In general, however, they are in the range from 1 to 65 hours, preferably 5 to 24 hours and especially preferably 5 to 22 hours. In the continuous processes, the residence times are generally in the range of 5 to 24 hours, preferably 5 to 22 hours.

The reaction can be carried out preferably while stirring, wherein the stirring speed is in the range of 50 to 2000 rpm and preferably in the range of 100 to 500 rpm.

The reaction is ideally carried out at standard pressure. The reaction temperature is between 20° C. and 80° C., preferably between 40° C. and 70° C.

The method according to the invention can be operated industrially, specifically both in continuous/semi-continuous mode and batchwise mode.

In comparison to the methods known from the literature, glycerol mono(meth)acrylate is obtained by the method according to the invention not only in anhydrous, but also in highly pure form.

The glycerol mono(meth)acrylate obtained by the method according to the invention can be used in applications which require a low residual water content or even the complete absence of water. In particular, it can be used in oil-based systems in the sector of lubricants and hydraulic oils, and in polymerization reactions in liquid and hydrophobic media.

The following examples illustrate the method according to the invention without these being limited thereto.

### Examples

Apparatus: 500 ml stirring apparatus, air inlet, Büchi rotary evaporator with vacuum accessories, pressure filter. Magnetic stirrer, oil pump.

Reaction:

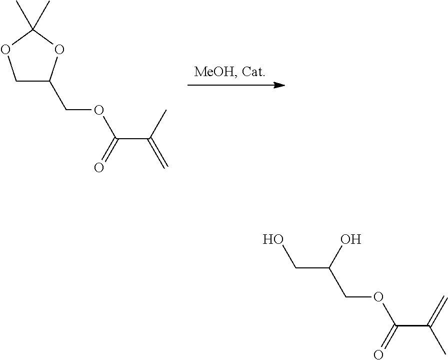

| | | | | |
|---|---|---|---|---|
| 50 | g | of 2,2-dimethyl-1,3-dioxolan-4-ylmethyl | 0.25 | mol |
| | | methacrylate (IPGMA) = | | |
| 80 | g | of methanol = | 2.5 | mol |
| 0.016 | g | of hydroquinone monomethyl ether | 400 | ppm |
| | | rel. to GMMA = | | |
| 0.004 | g | of hydroxy-2,2,6,6-tetramethylpiperidinooxyl | 100 | ppm |
| | | (TEMPOL); rel. to GMMA = | | |

| | | | |
|---|---|---|---|
| 13 | g | of catalyst/Tonsil ® 312 FF standard | 10% |
| | | rel. to mixture = | |

Mixture:

Procedure:

The batch is boiled at 65° C. under reflux with stirring for 20 h. The GMMA crude ester obtained is filtered through a pressure filter with K800 filter layer. The filter is rinsed with ca. 100 g of methanol. The clear, pale yellow GMMA crude ester is freed of solvent at 50° C. bath temperature at 20 mbar on a rotary evaporator for 30 min.

Results:

| | | | | | | | | GMMA GC-RV | | | | | | |
|---|---|---|---|---|---|---|---|---|---|---|---|---|---|---|
| | | | | % by | Reaction | | | in area % | | | | | | |
| | | | | weight | Dura- | Bottoms | Mass | | | | | Glycerol | | |
| | IPGMA | MeOH | | rel. to | tion | max. | g = % of | | | | | dimeth- | | |
| | mol | mol | Catalyst | mixture | h | ° C. | theory | MeOH | IPG | IPGMA | Glycerol | acrylate | GMMA | Remarks |
| 1 | 0.25 | 2.5 | Amberlyst ® | 10 | 20 | 50 | 43 g = | — | 9.3 | 0.4 | 16.4 | 16.5 | 35.9 | Clear |
| | | | A15 washed | | | | 38% | | | | | | | dark |
| | | | and dried | | | | | | | | | | | product |
| | | | LJ: 20896/57 | | | | | | | | | | | |
| 2 | 0.25 | 2.5 | p-TS | 1 | 22 | 50 | 43 g = | — | 26.5 | 2.9 | 25.2 | 8.9 | 7.8 | Clear |
| | | | | | | | 8% | | | | | | | yellow |
| | | | | | | | | | | | | | | product −> |
| | | | | | | | | | | | | | | polymer |
| 3 | 0.25 | 2.5 | Montmoril- | 10 | 22 | 50 | 41 g = | 3.0 | 1.0 | 34.2 | 0.8 | 0.1 | 56.6 | Clear |
| | | | lonite K10 | | | | 58% | | | | | | | yellow |
| | | | | | | | | | | | | | | product |
| 4 | 0.25 | 2.5 | Montmoril- | 10 | 5 | 50 | 41 g = | 2.3 | 0.6 | 37.4 | 0.5 | <0.1 | 58.0 | Clear |
| | | | lonite K10 | | | | 59% | | | | | | | yellow |
| | | | | | | | | | | | | | | product |
| 5 | 0.25 | 2.5 | Montmoril- | 10 | 5 | 65 | 42 g = | 0.1 | 1.1 | 26.1 | 1.6 | 0.1 | 65.3 | Clear |
| | | | lonite K30 | | | | 69% | | | | | | | yellow |
| | | | | | | | | | | | | | | product |
| 6 | 0.25 | 2.5 | Tonsil ® | 10 | 22 | 50 | 41 g = | 0.6 | 0.5 | 31.2 | 0.4 | — | 66.6 | Clear |
| | | | 312 FF | | | | 68% | | | | | | | pale |
| | | | | | | | | | | | | | | yellow |
| | | | | | | | | | | | | | | product |
| 7 | 0.25 | 2.5 | Tonsil ® | 10 | 64 | RT | 44 g = | 2.2 | 0.5 | 38.0 | 0.4 | — | 58.7 | Clear |
| | | | 312 FF | | | | 65% | | | | | | | pale |
| | | | | | | | | | | | | | | yellow |
| | | | | | | | | | | | | | | product |
| 8 | 0.25 | 2.5 | Tonsil ® | 10 | 20 | 65 | 43 g = | 2.3 | 0.5 | 22.3 | 0.6 | <0.1 | 73.1 | Clear |
| | | | 312 FF | | | | 79% | | | | | | | pale |
| | | | | | | | | | | | | | | yellow |
| | | | | | | | | | | | | | | product |

1-14. (canceled)

15. A method for producing glycerol mono(meth)acrylate, comprising reacting 2,2-dimethyl-1,3-dioxolan-4-ylmethyl (meth)acrylate with methanol in the presence of an acidic silicate catalyst.

16. The method of claim 15, wherein the silicate in said acidic silicate catalyst is a sheet silicate or a mixture of sheet silicates.

17. The method of claim 16, wherein the sheet silicate or the mixture of sheet silicates have a specific surface area greater than 50 m²/g.

18. The method of claim 16, wherein the sheet silicate or the mixture of sheet silicates have a specific surface area greater than 320 m²/g.

19. The method of claim 16, wherein the sheet silicate or the mixture of sheet silicates is selected from the group consisting of: montmorillonite; kaolinite; hectorite; halloysite; and bentonite.

20. The method of claim 16, wherein the sheet silicate or the mixture of sheet silicates is montmorillonite or bentonite.

21. The method of claim 15, wherein the reaction is performed in anhydrous manner and the product is anhydrous glycerol mono(meth)acrylate.

22. The method of claim 15, wherein the acidic silicate catalyst is present in an amount of from 1% by weight to 15% by weight, based on the reaction batch.

23. The method of claim 15, further comprising at least one stabilizer selected from the group consisting of: octadecyl-3-(3,5-di-tert-butyl-4-hydroxyphenyl) propionate; phenothiazine, hydroquinone; hydroquinone monomethyl ether; 4-hydroxy-2,2,6,6-tetramethylpiperidin-ooxyl (TEMPOL); 2,4-dimethyl-6-tert-butylphenol; 2,6-di-tert-butylphenol; 2,6-di-tert-butyl-4-methylphenol; para-substituted phenylenediamines; 1,4-benzoquinone; 2,6-di-tert-butyl-alpha-(dimethylamino)-p-cresol; and 2,5-di-tert-butylhydro-quinone.

24. The method of claim 23, wherein the total amount of stabilizer is between 0.001% by weight and 0.5% by weight.

25. The method of claim 23, wherein the stabilizers hydroquinone monomethyl ether and hydroxy-2,2,6,6-tetramethylpiperidinooxyl (TEMPOL) are used in combination.

26. The method of claim 25, wherein the ratio of hydroquinone monomethyl ether to TEMPOL is in the range of 10:1 and 4:1.

27. The method according of claim 15, wherein the reaction temperature is between 20° C. and 80° C.

28. The method of claim 27, wherein the reaction temperature is between 40° C. and 70° C.

29. Glycerol mono(meth)acrylate comprising a content of glycerol di(meth)acrylate of less than 1% and a content of glycerol of less than 1%.

30. The method of claim 18, wherein the sheet silicate or the mixture of sheet silicates is selected from the group consisting of: montmorillonite; kaolinite; hectorite; halloysite; and bentonite.

31. The method of claim 30 wherein the sheet silicate or the mixture of sheet silicates is selected from the group consisting of: montmorillonite and bentonite.

32. The method of claim 31, wherein the reaction is performed in anhydrous manner and the product is anhydrous glycerol mono(meth)acrylate.

33. The method of claim 31, wherein the acidic silicate catalyst is present in an amount of from 1% by weight to 15% by weight, based on the reaction batch.

34. The method of claim 33, further comprising at least one stabilizer selected from the group consisting of: octadecyl-3-(3,5-di-tert-butyl-4-hydroxyphenyl) propionate; phenothiazine; hydroquinone; hydroquinone monomethyl ether; 4-hydroxy-2,2,6,6-tetramethylpiperidin-ooxyl (TEMPOL); 2,4-dimethyl-6-tert-butylphenol; 2,6-di-tert-butylphenol; 2,6-di-tert-butyl-4-methylphenol; para-substituted phenylenediamines; 1,4-benzoquinone; 2,6-di-tert-butyl-alpha-(dimethylamino)-p-cresol; and 2,5-di-tert-butylhydro-quinone.
